(19) United States
(12) Patent Application Publication
(10) Pub. No.: US 2016/0157935 A1
(43) Pub. Date: Jun. 9, 2016

(54) DEVICE AND METHOD FOR NON-INVASIVE TREATMENT OF SKIN USING LASER LIGHT

(71) Applicant: KONINKLIJKE PHILIPS N.V., Eindhoven (NL)

(72) Inventors: MARTIN JURNA, EINDHOVEN (NL); JONATHAN ALAMBRA PALERO, EINDHOVEN (NL); BABU VARGHESE, EINDHOVEN (NL); MARGARET RUTH HORTON, EINDHOVEN (NL)

(21) Appl. No.: 14/903,634

(22) PCT Filed: Jul. 4, 2014
(86) PCT No.: PCT/EP2014/064301

§ 371 Date: Jan. 8, 2016

(30) Foreign Application Priority Data
Jul. 11, 2013 (EP) 13176059.7

(51) Int. Cl. A61B 18/20

(52) U.S. Cl. CPC A61B 18/203; A61B 2018/0047

(57) ABSTRACT

An increasing number of non-invasive skin treatment devices are being provided for use by consumers instead of by medical professionals. Such home use raises new concerns, such as safety and treatment efficacy. The invention improves on existing devices and treatment methods. The invention provides a device and method, wherein a light source is configured and arranged to provide a first (31) and a second (32) region of non-zero intensity within a transverse cross-section of a laser light beam (21) having a single higher-order laser beam mode, and a third region (33), disposed between the first (31) and second (32) regions, of lower light intensity than the non-zero light intensity. The first (31) and second (32) regions are configured to create, during use, a lesion in skin tissue in the focal spot of the laser light beam, and the third (33) region is configured to avoid creating in the focal spot, during use, a lesion in skin tissue between the lesions created by the first and second regions. For example, the light source may be configured and arranged to produce laser light in a higher mode, the order being higher than fundamental mode. The intensity pattern of the beam profile, with its regions of substantially differing intensity, is substantially constant along the propagation axis. This provides an area in the skin tissue in the focal spot at the treatment location with substantially no lesions, disposed between areas of lesions.

### FIELD OF THE INVENTION

The invention relates generally to the treatment of skin using laser light, and more particularly to a non-invasive device and method for treatment.

### BACKGROUND OF THE INVENTION

Various forms of electromagnetic radiation, particularly laser light beams, have been used for many years on skin for a variety of treatments, such as hair removal, skin rejuvenation to reduce wrinkles and reduction of pigmentation spots, and the treatment of conditions like acne, actinic keratoses, blemishes, scar tissue, discoloration, vascular lesions, acne treatment, cellulite and tattoo removal. Most of these treatments rely on photothermolysis, where a treatment location is targeted by the treatment radiation. The desire to maintain a youthful appearance by preventing or reducing wrinkles in the skin is an important issue in human society. Many non-invasive techniques have been designed to address the above issue, where the dermis layer is damaged by heating (thermolysis) to induce a wound response without damage to the epidermis. In other words, a target area within the skin is damaged in a controlled way, and the skin is allowed to replace the damaged tissue by renewed tissue—the damage promotes healing and a rejuvenation effect occurs. The renewed tissue improves the skin's radiance, tone, and can even provide a reduction in pore size, wrinkles and fine lines.

In the process of photothermolysis, heating is performed using electromagnetic radiation which can penetrate the skin to the dermal layer—for example, focused laser light may be used to create laser induced optical breakdown (LIOB) inside the dermis layer, as known from the published international patent application WO 2008/001284 A2.

Selective non-ablative photothermolysis based on the absorption of light by water is used to heat the tissue to a temperature ranging between 60-100 degrees Celsius to induce these damaged areas, without ablation or vaporization of the skin. Ablative photothermolysis occurs when the water temperature exceeds 100 degrees.

Initially, such rejuvenation treatments were applied to the full treatment area this is the most effective treatment with the best treatment outcome because all the tissue is damaged. However, the side effects are substantial, potentially permanent and the risk of complications is high.

Most current treatments are based upon fractional thermolysis, where isolated non-contiguous micro-thermal wounds (or lesions) are formed, creating necrotic zones surrounded by zones of viable tissue. This is further described in the published US patent application US 2005/0049582, and the article “Fractional photothermolysis: a new concept for cutaneous remodeling using microscopic patterns of thermal injury by Manstein D, Herron G S, Sink R K, et al.; Lasers in Surgery and Medicine 34:426-438 (2004). Such a fractional treatment has been shown to reduce social downtime significantly, whilst still providing good results.

US 2006/0020309 A1 discloses a method and device for the treatment of skin tissue with electromagnetic radiation (EMR) to produce lattices of EMR-treated islets in the skin tissue. In an embodiment the device has a single laser source and a phase mask including a set of apertures that spatially modulate the laser light beam received from the laser source. In this embodiment, the phase mask splits the laser light beam generated by the single laser source into a plurality of treatment laser light beams that are each focused by an optical element into a separate focal spot in the skin tissue. The pattern of focal spots of the plurality of treatment laser light beams creates a pattern of isolated lesions (lattices) in the skin tissue.

US 2009/0254073 A1 discloses a dermatological treatment device for generating a matrix of two dimensional treatment spots on the tissue. In this device, a diffractive element is positioned between a single laser source and a lens system to spatially modulate, i.e. to split the laser beam received from the laser source into a plurality of sub-beams. Also in this device, each sub-beam is focused by the lens system into a separate focal spot. Similar devices using spatially modulating diffractive optical elements to split a laser light beam into a plurality of treatment laser beams impinging on the skin are disclosed by WO 2008/089344 and WO 2004/000419.

An increasing number of these non-invasive skin treatment devices are being provided for use by consumers instead of by medical professionals. Such home use raises new concerns, such as safety and treatment efficacy. This is particularly important when the light source is a laser, and incorrect operation can result in scarring or burning of the skin where the laser light passes through the skin layers. It is desirable to improve the efficacy of such treatments by an optimal choice of relevant parameters, which include lesion diameter, lesion depth, coverage, healing time and thermal distribution of energy within the lesion.

### SUMMARY OF THE INVENTION

An object of the invention is to provide an improved non-invasive skin treatment device and method using laser light.

The object is achieved according to the invention by a device comprising:
a light source for generating a laser light beam;
an optical system configured and arranged to receive the laser light beam from the light source, and further configured:
   to guide, in use, the laser light beam so as to exit the device as a treatment laser beam which impinges on an outer surface of the skin to be treated, and
   to focus, in use, the treatment laser beam to a single focal spot corresponding to a treatment location in skin tissue;
      wherein the light source is further configured and arranged to provide a single higher-order laser beam mode, whereby a transverse cross-section of the laser light beam has:
at least a first and a second region of non-zero light intensity, and
a third region, disposed between the first and second regions, of lower light intensity than the non-zero light intensity;
   wherein the first and second regions are each configured to create, during use, a lesion in skin tissue in the focal spot, and wherein the third region is configured to avoid creating in the focal spot, during use, a lesion in skin tissue between the lesions created by the first and second regions.

The object is also achieved by a non-invasive method of treating skin using a device generating laser light, the device comprising a light source for generating a laser light beam, and an optical system configured and arranged to receive the laser light beam from the light source;

the method comprising:
generating the laser light beam using the light source;
guiding the laser light beam through the optical system so as to exit the device as a treatment laser beam which impinges on an outer surface of the skin to be treated, and
focusing the treatment laser beam, using the optical system, to a single focal spot corresponding to a treatment location in skin tissue;
   wherein the method further comprises configuring and arranging the light source to provide a single higher-order laser beam mode, whereby a transverse cross-section of the laser light beam has:
at least a first and a second region of non-zero light intensity, and
a third region, disposed between the first and second regions, of lower light intensity than the non-zero light intensity;
   wherein the first and second regions are each configured to create a lesion in skin tissue in the focal spot, and wherein the third region is configured to avoid creating in the focal spot a lesion in skin tissue between the lesions created by the first and second regions.

By providing a single higher-order laser beam mode, the light source is configured and arranged to provide regions of differing intensity within the transverse cross-section of the laser light beam. When viewed in the transverse cross-section, the laser light beam has a first and second region of non-zero intensity and a third region of substantially lower intensity between them. By virtue of the single higher-order laser beam mode, the beam profile with its regions of substantially differing intensity is substantially constant along the propagation axis up to the single focal spot of the laser light beam. The focal spot is configured and arranged to provide treatment radiation at a treatment location corresponding to the position of the focal spot. For example, the treatment location may be on the axis of the treatment beam within skin tissue, whereas the focal spot may be on or above the surface of the skin. There is a region in the transverse cross-section of the focal spot with a significantly lower energy that corresponds to the third region in the beam profile. This provides a substantially lesion-free area in the skin tissue at the treatment location in the focal spot, which substantially lesion-free area is disposed between areas of lesions in the focal spot.

The presence of skin tissue areas without lesions proximate to skin tissue areas with lesions provides a reduced healing time for the skin tissue with lesions. By virtue of the regions of differing intensity in the transverse cross-section of the laser light beam provided by the single higher-order laser beam mode, the possible lesion length within the skin, seen in the propagation direction of the laser light beam, is considerably increased, allowing treatment at deeper locations below the skin surface or over a longer range of depths within the skin tissue. This increased lesion length is a result of the increased Rayleigh length of the focal spot, which results from the use of the single higher-order laser beam mode. This may also have the advantage that the treatment areas with lesions and without lesions are separated by a deeper channel of healthy tissue, providing a more precise separation of lesion regions.

Preferably, the regions are configured to create lesions in the skin tissue separated by at least one layer of skin cells by selecting parameters such as position, dimensions and intensity. At least one layer of skin cells is believed to be advantageous in providing the healing effect for the area with skin lesions.

The number of regions of non-zero intensity may be most advantageous when it is an integer in the range of 2 up to and including 24.

It may be advantageous for the light source to comprise a laser source configured and arranged to oscillate in a transverse mode having a higher order than the fundamental mode of the laser source, wherein the first, second and third regions form part of the intensity pattern of the transverse mode within the transverse cross-section of the laser light beam.

This provides a relatively simple implementation of the invention, because any suitable technique known in the art may be used to configure the laser source to operate in a transverse mode that is not the fundamental mode of the laser source. These transverse modes are typically called higher order transverse modes, being of a higher order than the fundamental mode. The fundamental mode of the laser source is typically designated as the TEM00 or LG00 or HG00 mode. The higher order transverse modes have a well-defined and regular intensity pattern in the transverse cross-section of the laser light beam. The invention uses part, or even all, of this intensity pattern to provide the regions of non-zero and lower light intensity. These transverse modes may, for example, be rectangular, cylindrical, or elliptical transverse modes, wherein the single higher-order laser beam mode is, for example, a Hermite-Gaussian mode, a Laguerre-Gaussian mode, or an Ince-Gaussian mode.

It may be also advantageous for the light source to further comprise at least one phase-modulating optical element configured and arranged to provide an equivalent of the single higher-order laser beam mode. This phase-modulating optical element (or optical elements) may be disposed within the laser cavity and/or outside the laser cavity. Any suitable phase-modulating optical element known in the art may be used, for example, a Spatial Light Modulator (SLM), a Diffractive-Optical element (DOE), a phase mask, a spiral wave plate, a vortex wave plate, a Pitch-Fork Hologram, a Q-Plate, or a Cylindrical Mode Converter. It may be advantageous to combine more than one of these phase-modulating optical elements to provide the regions of non-zero and lower light intensity in the transverse cross-section laser light beam. It may also be advantageous to accommodate at least one phase-modulating optical element inside the laser cavity, in combination with at least one optical element outside the laser cavity.

When the light source comprises a laser cavity and the at least one phase-modulating optical element is provided outside the laser cavity, it may be advantageous to configure the laser cavity to operate in the Gaussian fundamental mode. This provides a predetermined light source for the phase-modulating optical element, which may simplify the design of the phase-modulating optical element.

Although the invention may provide many configurations of the regions of non-zero light intensity and lower light intensity in the transverse cross-section of the laser light beam, it may be particularly advantageous to provide first and second regions of non-zero light intensity that are non-contiguous, and to configure the third region of lower light intensity so as to separate the first and second regions along the total length of their borders within the transverse cross-section of the laser light beam. These separated first and second regions provide separate regions within the focal spot. In other words, the focal spot is divided into sub-spots, allowing smaller spot sizes to be achieved compared to the focal spot size of a Gaussian beam profile.

### BRIEF DESCRIPTION OF THE DRAWINGS

FIG. 1 diagrammatically shows the most important parts of an operational skin treatment device 10 according to the invention, when treating skin 15,

FIG. 2 and FIG. 3 show examples of the transverse cross-section of the laser light beam 21 of the device according to the invention, comprising regions of non-zero light intensity 31, 32 and a region of lower light intensity 33;

FIG. 4 depicts examples of rectangular transverse laser modes that the laser light source of the device according to the invention may provide, for example, Hermite-Gaussian modes;

FIG. 5 depicts examples of cylindrical transverse laser modes that the laser light source of the device according to the invention may provide, for example, Laguerre-Gaussian modes;

FIG. 6A schematically depicts a longitudinal cross-section of the focal spot 22, using a laser light source known in the art;

FIG. 6B schematically depicts a longitudinal cross-section of the focal spot 22 using a laser light source in a device according to the invention, where the laser light source 20 is operated in LG01* or “doughnut” mode, and

FIG. 7 depicts a further embodiment of a device according to the invention having a laser light source and at least one phase-modulating optical element 122 configured and arranged to provide an equivalent of a single higher-order laser beam mode.

It should be noted that items which have the same reference numbers in different Figures, have the same structural features and the same functions, or are the same signals. Where the function and/or structure of such an item has been explained, there is no necessity for repeated explanation thereof in the detailed description.

### DETAILED DESCRIPTION OF EXAMPLES

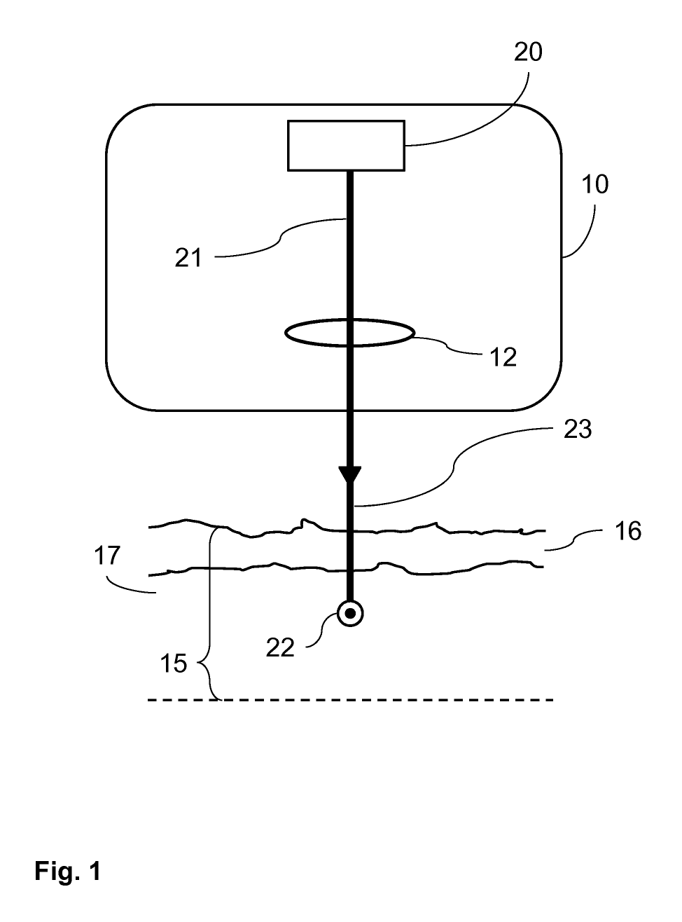

FIG. 1 schematically shows a skin 15 treatment device 10 according to the invention, comprising a light source 20 for generating a laser light beam 21 and an optical system 12, configured and arranged to receive the laser light beam 21. The laser light beam 21 has a suitable frequency and pulse duration for treating human or animal skin 15. The light source 20 is typically a pulsed laser—for example a Nd:YAG laser with emission at 1064 nm and 1-1000 ps pulse duration. During use, the device (10) emits the light as a treatment laser beam 23 which is focused to a single focal spot 22 corresponding to a treatment location in the skin 15.

The skin 15 comprises multiple layers with different optical properties. The epidermis 16 is composed of the outermost layers and forms a waterproof protective barrier. The outermost layer of the epidermis 16 is the stratum corneum which, due to its microscopic fluctuations in roughness, impedes the coupling of light between the device 10 and the skin 15. Typically an index-matching fluid is used to optically couple the treatment laser beam 23 into the skin 15. The dermis 17 is situated underneath the epidermis 16—the focal spot 22 is typically disposed in the dermis 17 for skin treatments. If the device 10 is used to reduce wrinkles in the skin 15, the treatment location is in the collagen of the dermis 17 in order to create microscopic lesions at the position of the treatment location, which results in new collagen formation.

The focal spot 22 is configured and disposed to provide treatment radiation at a treatment location. The spatial relationship between the focal spot 22 and the treatment location may depend on the intensity profile of the transverse beam cross-section, the nature of the skin tissue present between the focal spot 22 and the treatment location, the depth of the treatment location beneath the surface of the skin and the optical properties of the treatment beam 23. The focal spot 22 may even be on or above the surface of the skin corresponding to a treatment location on or beneath the skin surface. In many cases, the focal spot 22 may coincide with the treatment location.

If the treatment location is collagen within the skin, the focal spot 22 may be disposed between 0.2 and 2 mm below the outer surface of the skin 15, in particular between 0.5 and 1.5 mm below the outer surface of the skin 15.

The optical system 12 is configured and arranged to guide, in use, the laser light beam 21 to exit the device 10 as a treatment laser beam 23 which impinges on an outer surface of the skin 15 to be treated, and to focus, in use, the treatment laser beam 23 to the single focal spot 22 corresponding to a treatment location in the skin. The word “guide” includes configurations where the direction of the light beams is substantially changed, and configurations where the light beams 21, 23 are allowed to propagate along the light beam direction without substantial change, as well as any intermediate degree of directional change.

The device 10, and in particular the optical system 12, are configured to produce photothermolysis at the treatment location by selecting or modifying at least one optical property of the laser light beam 21 to provide a treatment laser beam 23. The convergence of the treatment beam 23 into the focal spot 22 helps localize the phenomenon and helps prevent damage to the epidermis 16 when the focal spot 22 is disposed beneath the skin, because there the power density is much lower than in the dermis 17.

The invention uses the fact that the skin transmits electromagnetic radiation. To maximize this effect, the wavelength of the light is between 800 and 1100 nm. In this range, transmission is high and scattering and linear absorption are low. It is however not excluded to use other wavelengths.

Typically, the deliverable energy level in the laser beam pulse is between 0.1 and 20 mJ, measured at the surface of the skin. Such energy levels have turned out to be useful in skin treatment. In the above energy level indications, the energy is measured at the surface of the skin, i.e. it relates to the energy actually delivered into the skin.

The optical system 12 may comprise at least one lens for converging and/or diverging the laser light beam 21, and at least one mirror for deflecting the laser light beam in a desired direction to provide a treatment laser beam 23. The exact position and/or orientation of the optical elements may be adjustable using techniques known in the art to adapt the position and quality of the focal spot 22 to what is required for the treatment being performed. Focus control may be provided by adjusting the position of at least one of the lenses and/or rotating at least one of the mirrors. The number and positions of lenses and mirrors are determined by the disposition of the components within the optical system 12 and the desired degrees of adjustment that the skilled person wishes to provide. The optical system 12 may further comprise a laser beam manipulator for positioning the focal spot 22. Such a laser beam manipulator may comprise, for example, a moveable mirror and a mirror actuator for adjusting the position of the mirror, as well as a control unit. The laser beam manipulator may also comprise the adjustable lens or mirror. The laser beam manipulator may be used for positioning the focal spot on and in the skin 15. The user may control the laser beam manipulator.

The laser light beam 21 that is received by the optical system 12 has a single higher-order laser beam mode, resulting in a non-Gaussian transverse profile of the laser light beam 21. FIG. 3 shows an example of the transverse cross-section of the laser light beam 21, comprising a first and second region 31, 32 of a non-zero light intensity and a third region 33 of a lower light intensity than the non-zero light intensity. The third region 33 has a substantially lower light intensity than the first and second regions 31, 32. The term “substantially” used here means that the intensity of the first and second regions 31, 32 is configured to create in the focal spot 22, during use, a lesion in the skin tissue at the treatment location corresponding to the focal spot 22, and that the intensity of the third region 33 is configured to avoid creating in the focal spot 22, during use, a lesion in the skin tissue at the treatment location corresponding to the focal spot 22.

The first and second regions 31, 32 of FIG. 3 are contiguous, forming an annular region of non-zero intensity. At the center of the annulus is the third region 33 with a substantially lower intensity. To avoid creating lesions in the skin 15 during use, the intensity in the third region 33 may be in the range of zero intensity up to an intensity threshold. This intensity threshold corresponds to the maximum energy level in the skin tissue which causes no lesions. This maximum energy level depends upon the position and dimensions of the regions, the type of skin treatment, the depth of the treatment location in the skin, and the treatment laser beam properties such as wavelength spectrum, spot width, pulse length, and irradiance. The intensity threshold may also depend on where the skin to be treated is located on the body, the individual being treated, and the phase in the treatment cycle.

The intensity in the first region 31 and in the second region 32 are above this intensity threshold, and the dimensions of the first and the second region 31, 32 and the intensity of the treatment light in the first and the second region 31, 32 are configured to create at least one lesion in the skin 15.

The intensity in the first and second regions 31, 32 may also vary. In practice, each region may have an approximately Gaussian profile, and the edge of each region 31, 32 may be considered to be a contour joining points of intensity equal to half the maximum intensity within the region.

A single higher-order laser beam mode, resulting in a transverse cross-section of the laser beam 21 having regions of non-zero and lower light intensity, may be provided by configuring and arranging the laser light source 20 to operate in a higher order mode in other words, not in the fundamental mode and not in a mixed multi-mode where the output is a combination of a plurality of higher-order modes. The higher order mode should be pure enough to create the required regions of non-zero and substantially lower light intensity. Any methods known in the art may be used to achieve this, such as appropriate configuration of the laser cavity or resonator. For example, the article “Generation of pure TEMp0 modes using a friendly intra-cavity laser beam shaping technique” by Cagniot, Fromager et al; Laser Beam Shaping XII; SPIE Vol. 8130, 813006 (2011) discloses a model for generating higher modes with phase and amplitude DOEs in a laser cavity (resonator). For example, a pi-phase plate is inserted into a plano-concave cavity. In particular, results from the generation of the Laguerre-Gaussian (LG) transverse modes TEM-10, TEM-20 and TEM-30 are depicted in FIG. 8 of that article. In this patent application, the standard notation for LG modes is used—the first index indicates the number of radial mode orders (p), and the second index indicates the number of angular mode orders (1).

The intensity regions of FIG. 3 may be provided by configuring and arranging the laser light source 20 to operate in the LG-01* mode, where p=0 and l=1. The “*” indicates that this is the so-called “doughnut” mode, where the non-zero intensity region is an annulus surrounding a low-intensity region.

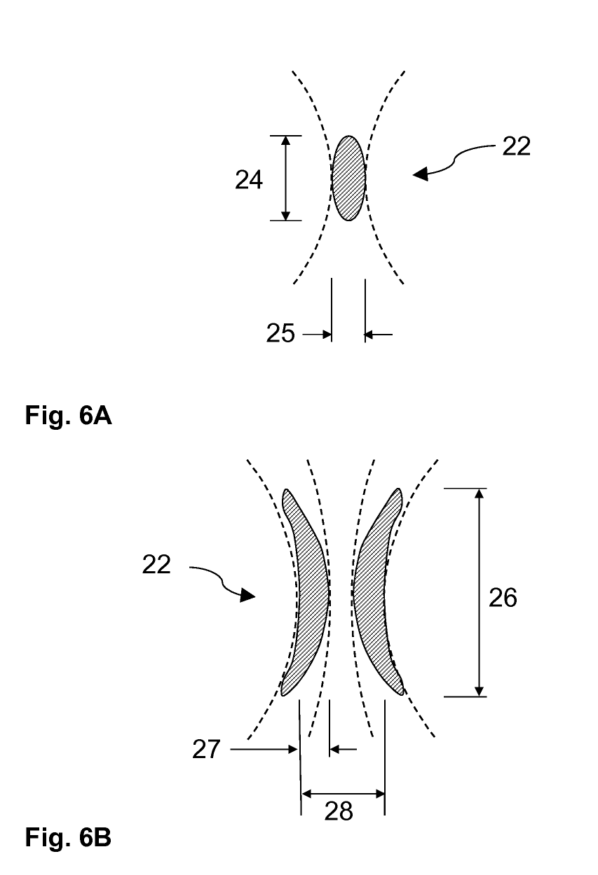

FIG. 6B depicts schematically a longitudinal cross-section of the focal spot 22 using a laser light source according to the invention, where the laser light source 20 is operated in LG01* or “doughnut” mode. As the intensity regions are provided by a single higher-order laser beam mode of the light source 20, the regions are substantially constant along the propagation axis of the laser light beam 21, including at the locations within the skin and within the focal spot 22. The treatment laser beam 23 (not depicted) propagates along the same longitudinal axis. The intensity profile in the transverse cross-section is therefore also an annulus of non-zero intensity surrounding a central region of lower intensity. FIG. 6B depicts two crescent-shaped areas of non-zero intensity, extending in the longitudinal direction. When the treatment location coincides with the focal spot 22, the intensity depicted is the intensity in the skin 15 which causes a lesion—it is here assumed that the intensity threshold in the skin is the same as the half-maximum level, corresponding with the FWHM spot measurement. The spot width 28 of the focal spot 22 is measured in the transverse direction from a first position on the outer contour of the non-zero intensity region to a second position on the outer contour, the first and second positions both lying in the transverse cross-section, symmetrically about the longitudinal axis. This spot measurement 28 corresponds to the FWHM measurement including all mode orders.

In longitudinal cross-section, a region of lower intensity is disposed at the center of the transverse cross-section, extending along the propagation axis. The invention is based on the insight that in the higher order laser modes, each region of non-zero intensity may be considered to be a sub-spot, with a sub-spot width 27 measured in the transverse direction from a third position on the outer contour of the sub-spot to a fourth position on the outer contour of the sub-spot, the third and fourth positions both lying in the transverse cross-section.

The distance 26 along which each sub-spot extends longitudinally (i.e. in the propagation direction of the laser light beam) is assumed to correspond to the depth of the lesions in the skin at the treatment location.

For comparison, FIG. 6A depicts schematically the longitudinal cross-section of the focal spot 22 using a laser light source, where the laser light source 20 is operated in fundamental mode. The beam profile is assumed to be Gaussian or approximately Gaussian. The intensity profile in the transverse cross-section is therefore also a circle of non-zero intensity extending over the whole spot 22. The region of intensity extends 24 in the longitudinal direction. The spot width 25 of the focal spot 22 is measured in the transverse direction from a first position on the outer contour of the non-zero intensity region to a second position on the outer contour, the first and second positions both lying in the transverse cross-section, symmetrically about the longitudinal axis. This spot measurement 25 corresponds to the FWHM measurement over the whole spot 22.

For comparison, it is assumed that the longitudinal length (or depth) of the lesion in the skin tissue is directly related to the value of the Rayleigh length. For FIG. 6A, the Rayleigh length 24 for a Gaussian beam z_{G} with a beam spot size 25 of ω_{G} and wavelength λ is given by:

$${z}_{G} = \frac{π {ω}_{G}^{2}}{λ}$$

The beam spot size of higher order Laguerre-Gaussian beams is \sqrt{2p+l+1} times broader than the TEM-00 beam, and the Rayleigh length of the higher order modes for a certain laser configuration is the same as the lowest order mode. So, for FIG. 6B, LG-01* (p=0, l=1), the beam spot size 28 is \sqrt{2} times that of the TEM-00. The Rayleigh length 26 for a LG-01* beam z_{LG01} with a beam spot size 28 of ω_{LG01} is given by:

$${z}_{LG 01} = \frac{{π ( \frac{1}{\sqrt{2}} {ω}_{LG 01} )}^{2}}{λ}$$

LG-01* in longitudinal cross-section comprises two crescent-shaped regions, which may be considered sub-spots. The sub-spot width 27 δ_{LG01}, derived from the intensity profile of LG01*, is 0.35ω_{LG01}.

The invention is based upon the insight that creating a required spot size in the skin may be performed using a region of the spot (a sub-spot) instead of the full spot. If the TEM-00 beam spot size 25 is made equal to the sub-spot size 27 in the LG01* mode (i.e. ω_{G}=δ_{LG01}), the ratio between the Rayleigh lengths of the LG-01* beam of FIG. 6B to the TEM-00 beam of FIG. 6A becomes:

$$\frac{{z}_{LG 01}}{{z}_{G}} = \frac{{( \frac{1}{\sqrt{2}} )}^{2} {ω}_{LG 01}^{2}}{{ω}_{G}^{2}} = \frac{{( \frac{1}{\sqrt{2}} )}^{2} {ω}_{LG 01}^{2}}{{( 0.35 )}^{2} {ω}_{LG 01}^{2}} = 4.1$$

Thus, for a given lesion width, the maximum lesion length (or lesion depth within the skin) created using LG-01 is 4.1 times longer than when a conventional TEM00 Gaussian beam is used, depending on the optical penetration depth of the laser.

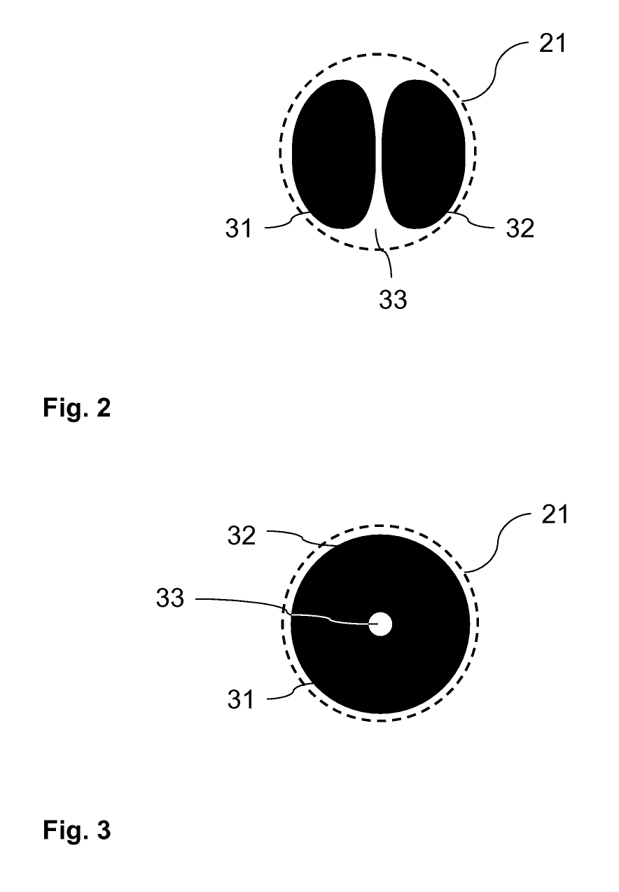

The same advantage is also achieved with other higher-order modes. For example, FIG. 2 shows a further example of the transverse cross-section of the laser light beam 21, comprising a first and a second region 31, 32 of non-zero intensity and a third region 33 of lower intensity. The non-zero intensity regions are non-contiguous, and have the appearance of two lobes, disposed symmetrically within the transverse cross-section of the laser light beam 21. The beam profile of FIG. 2 may be provided by configuring and arranging the laser light source 20 to operate in the Hermite-Gaussian modes HG-mn, where m=0 and n=1 or m=1 and n=0.

For HG-01 or HG-10, it can be shown that δ_{HG01}=0.35ω_{HG01}, and using the beam spot size (in the x-direction) of higher order Hermite-Gaussian being \sqrt{2m+1} times broader than TEM-00, the Rayleigh length is:

$${z}_{HG 01} = \frac{{π ( \frac{1}{\sqrt{3}} {ω}_{LG 01} )}^{2}}{λ}$$

and the ratio of the Rayleigh lengths of the HG-01 beam to the TEM-00 beam is

$$\frac{{z}_{HG 01}}{{z}_{G}} = 2.7$$

The advantage is achieved at different numerical apertures. In some treatments, the treatment laser may be focused such that the focal spot 22 is disposed on the surface of the skin—in that case, half of the Rayleigh length becomes significant for lesion-depth estimation. The table below shows the calculated improvement in the Rayleigh length, using a laser wavelength of 1064 nm for different numerical apertures (NA):

| | Lesion | Half-Rayleigh length (mm) | | |
|---|---|---|---|---|
| | width | | LG- | |
| NA | (μm) | TEM-00 | 01* | HG01 |
| Low (NA = 0.01) | 33.9 | 1.69 | 6.94 | 4.57 |
| Medium (NA = | 11.3 | 0.19 | 0.77 | 0.51 |
| 0.03) | | | | |
| High (NA = 0.1) | 3.4 | 0.02 | 0.07 | 0.05 |

More details on intensity distributions in higher-order laser modes can be found in Chapter 11: Laser Beam Diagnostics in a Spatial Domain by Tae Moon Jeong and Jongmin Lee, in the book Laser Pulse Phenomena and Applications, edited by F. J. Duarte, ISBN 978-953-307-405-4.

The simplest implementation may be achieved by configuring and arranging the light source 20 so as to oscillate in a transverse mode higher than the fundamental mode. Extensive experience and knowledge exists for these modes. The first, second and third regions 31, 32, 33 then form part of the transverse mode intensity pattern within the cross-section of the light beam 21. These transverse modes may be, for example, rectangular or cylindrical transverse modes.

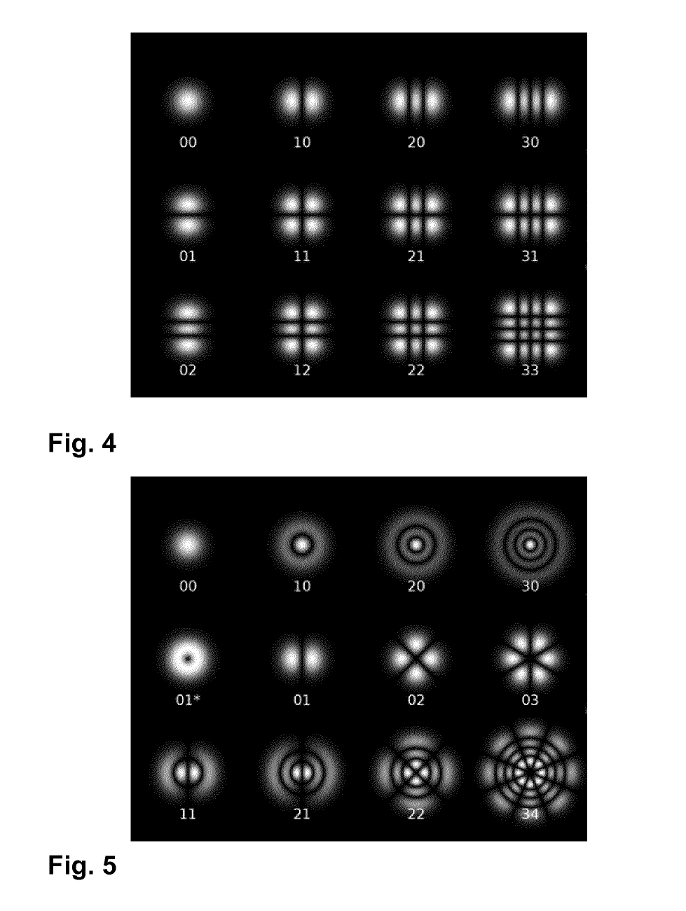

FIG. 4 depicts examples of known Hermite-Gaussian modes that may be used, such as HG-01, HG-02, HG-10, HG-11, HG-12, HG-20, HG-21, HG-22, HG-30, HG-31 and HG-32. For comparison, the fundamental mode HG-00 is also depicted with a single region of non-zero intensity. The skilled person will realize that any rectangular higher-order may be used.

FIG. 5 depicts examples of known Laguerre-Gaussian modes that may be used, such as LG-01*, LG-11, LG-10, LG-01, LG-21, HG-20, LG-02, LG-22, LG-30, LG-03 and LG-34. For comparison, the fundamental mode LG-00 is also depicted with a single region of non-zero intensity. The skilled person will realize that any cylindrical higher-order may be used.

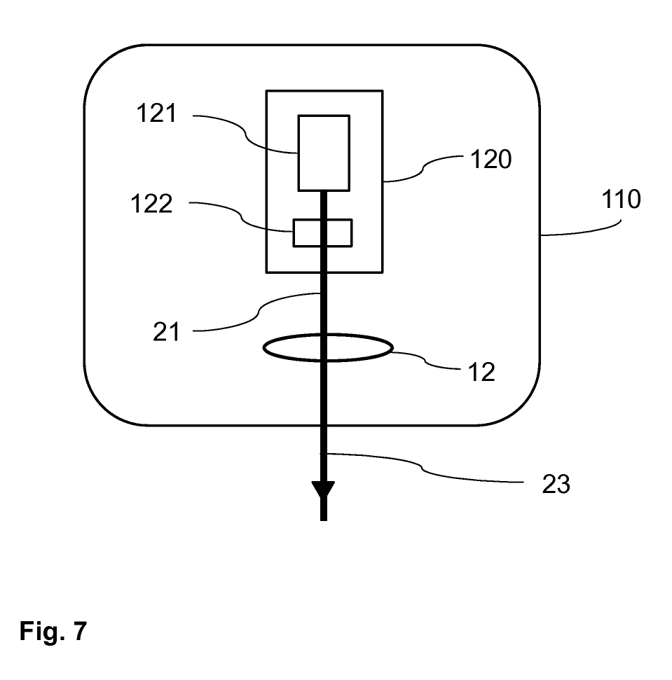

Configuring and arranging the laser source to oscillate in a substantially pure transverse mode is not the only way to provide the non-zero intensity and lower intensity regions in the laser light beam 21. A further alternative is depicted in FIG. 7, which illustrates a skin 15 treatment device 110 according to the invention, comprising a light source 120 for generating a laser light beam 21 and an optical system 12 configured and arranged to receive the laser light beam 21. The light source 120 comprises a laser cavity or resonator 121, which generates the laser light, and at least one phase-modulating optical element 122 configured and arranged to receive the laser light from the cavity 121 and to provide an equivalent of a single higher-order laser beam mode to the laser light beam 21 in order to provide the transverse cross-section of the laser light beam 21 with the first, second and third regions 31, 32, 33.

Any suitable optical phase-modulating elements known in the art may be used, for example, a Spatial Light Modulator (SLM) as known from U.S. Pat. No. 7,961,371, a Diffractive-Optical element (DOE), a phase mask as known from U.S. Pat. No. 7,982,938, a spiral wave plate, a vortex wave plate, a Pitch-Fork Hologram, a Q-Plate, or a cylindrical Mode Converter. A plurality and/or a combination of such elements may also be used. Equivalents or approximate equivalents to the LG modes may be provided using spiral or vortex wave plates, a Pitch-Fork Hologram, a Q-Plate and/or cylindrical Mode Converters. These phase-modulating optical elements should be configured and arranged to preserve the mode regions within the required Rayleigh length.

For example, the article “Creation of Laguerre-Gaussian laser modes using diffractive optics” by Kennedy, Szabo et al; Physical Review A 66, 043801 (2002) discloses the production of LG modes using diffractive optics outside of the laser cavity—these modes can propagate with minimal distortions over distances of 200 mm.

It may further be advantageous to operate the laser cavity 121 in the Gaussian fundamental mode. This provides a predetermined and regular light source for the phase-modulating optical element 122, which may simplify the design of the phase-modulating optical element 122.

A still further alternative is to combine both approaches—configure and arrange the laser cavity (resonator), and also incorporate at least one phase-modulating optical element in the light source 20, 120 disposed outside the cavity.

Until now the use of single higher-order laser beam modes has not been considered in these applications where small focal spot diameters combined with relatively large focal spot lengths (or depths) are desirable—the skilled person avoids higher order laser beam modes because of the increased difficulty to focus the beam to a small spot. This invention is based on the insight that the advantage of an increased lesion length or depth, due to the increased Rayleigh length, for a certain lesion width, provides a laser beam profile that has an unexpected advantage. The invention differs from known fractional techniques, such as described in the published US patent application US 2005/0049582, because the areas of healthy tissue proximate lesioned tissue are created at the treatment location by intensity distributions within the single focal spot 22 over an increased length or depth.

Providing a single focal spot 22 with regions of lower intensity between regions of non-zero intensity may also be advantageous because the areas of one or more lesions created at the treatment location within a single pulse event will be more temperature homogeneous within the individual lesions. Areas of low intensity between the sub-spots may limit thermal diffusion between the different areas treated by the sub-spots, and provide a more predictable thermal relaxation time and less cross-talk. By using sub-spots, better controllability of lesion outcome and uniform energy deposition can be achieved within the treatment region.

It may be advantageous to cause the lesions in the skin tissue at the treatment location to be separated by at least one layer of skin cells to have an appropriate degree of “free edges”. For skin treatments using laser beams in the fundamental mode, the focal spot 22 is typically less than 200 micron, preferably less than 50 micron. This invention, through its use of sub-spots, allows even smaller spots to be used for skin treatment. However, very small sub-spots may be ineffective for skin treatment because evidence suggests that the greater the amount of healthy tissue (i.e. tissue without lesions) surrounding the tissue with lesions, the greater the reduction of the side effects. Additionally, too much healthy tissue will reduce the efficacy of the fractionated treatment.

Some research, such as disclosed in the article “Free edges in epithelia as cues for motility”, Cell Adhesion & Migration 5:2, 106-110; March/April 2011; (2011) Landes Bioscience, suggests that a higher degree of “free edges” around tissue areas with lesions may improve the healing time.

For example, the invention may be used to create photothermolysis in the epidermis, to improve radiance and skin tone by inducing necrosis of epidermal cells. The smallest useful separation between regions of lesions at the treatment location may be approximately the size of epidermal cells, which is 10-20 micron. The skilled person will realize that the invention may be configured to provide regions in the laser light beam 21 profile that are equivalent or approximately equivalent to the TEM-22, for example HG-22 and LG-22. When the optical system 12 is configured and arranged to provide a 50 micron focal spot, then the sub-spot will be between 10 and 20 micron in width when measured in transverse cross-section at the focal spot 22. The lesions at the treatment location will consequently have corresponding dimensions.

To achieve the same size of sub-spot dimension when using the highest order, or in a configuration in which the laser light beam 21 has too small a diameter, it may be advantageous for the optical system 12 and/or the laser light source 20, 120 to further comprise additional optical components to increase the transverse cross-sectional diameter of the beam. Any suitable technique for beam expansion, known to the skilled person, may be used.

The invention provides a means of creating deep lesions inside skin. Such a device and method may be used for any suitable treatment of skin, in particular non-invasive wrinkle reduction in the skin, reduction of actinic keratoses, scar tissue or acne and reduction of pigmentation spots. The device and method may be used for selective photothermolysis, in particular for ablative and non-ablative techniques.

The laser light source 20 may be disposed outside of the device 10, and connected to the device by means of an optical fiber. In this manner, a small and lightweight applicator unit is provided, with the bulkier and heavier laser source etc., in a separate and stationary unit.

It should be noted that the above-mentioned embodiments illustrate rather than limit the invention, and that those skilled in the art will be able to design many alternative embodiments.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

### REFERENCE NUMBERS

10 (Skin) treatment device
12 Optical system
15 Skin
16 Epidermis layer of skin
17 Dermis layer of skin
20 Light source for generating laser light beam (21)
21 Laser light beam
22 Focal spot corresponding to a treatment location in skin tissue
23 Treatment laser beam
24 Rayleigh length for fundamental mode
25 Spot size FWHM for fundamental mode
26 Rayleigh length for LG01 mode
27 Spot size—FWHM—for LG01 region of non-zero intensity
28 Spot size FWHM for LG01
31 First region of non-zero intensity
32 Second region of non-zero intensity
33 Third region of lower intensity
120 Second embodiment of light source (20)
121 Laser cavity
122 Optical element outside laser cavity

1. A non-invasive device for treatment of skin using laser light, the devices comprising:
a light source for generating a laser light beam;
an optical system configured and arranged to receive the laser light beam from the light source, and further configured:
to guide, in use, the laser light beam so as to exit the device as a treatment laser beam which impinges on an outer surface of the skin to be treated, and
to focus, in use, the treatment laser beam to a single focal spot corresponding to a treatment location in skin tissue;
wherein the light source is further configured and arranged to provide a single higher-order laser beam mode, whereby a transverse cross-section of the laser light beam has:
at least a first and a second region of non-zero light intensity, and
a third region, disposed between the first and second regions, of lower light intensity than the non-zero light intensity;
wherein the first and second regions are each configured to create, during use, a lesion in skin tissue in the focal spot, and wherein the third region is configured to avoid creating in the focal spot, during use, a lesion in skin tissue between the lesions created by the first and second regions.

2. A device according to claim 1, wherein the light source comprises a laser source configured and arranged to oscillate in a transverse mode having a higher order than the fundamental mode of the laser source, wherein the first, second and third regions form part of the intensity pattern of the transverse mode within the transverse cross-section of the laser light beam.

3. A device according to claim 2, wherein the single higher-order laser beam mode is a Hermite-Gaussian mode, a Laguerre-Gaussian mode, or an Ince-Gaussian mode, and wherein the transverse mode is a rectangular, cylindrical, or elliptical transverse mode.

4. A device according to claim 1, wherein the light source further comprises at least one phase-modulating optical element configured and arranged to provide an equivalent of the single higher-order laser beam mode.

5. A device according to claim 4, wherein the at least one phase-modulating optical element is selected from the list consisting of: Spatial Light Modulator (SLM), Diffractive-Optical element (DOE), phase mask, spiral wave plate, vortex wave plate, Pitch-Fork Hologram, Q-Plate, Cylindrical Mode Converter.

6. A device according to claim 5, wherein the light source comprises a laser cavity configured to operate in the Gaussian fundamental mode, and the at least one phase-modulating optical element is disposed outside the laser cavity.

7. A device according to claim 1, wherein the first, second and third regions are configured to create lesions in the skin tissue separated by at least one layer of skin cells.

8. A device according to claim 1, wherein a dimension of the focal spot is less than 200 microns, preferably less than 50 microns.

9. A device according to claim 1, wherein the first and second regions are non-contiguous, and the third region is configured to separate the first and second regions throughout the length of their borders within the transverse cross-section of the laser light beam.

10. A device according to claim 1, wherein the light source is configured and arranged to provide a number of regions of non-zero light intensity, the number being an integer in the range of 2 up to and including 24.

11. A non-invasive method of treating skin using a device generating laser light, the device comprising a light source for generating a laser light beam, and an optical system configured and arranged to receive the laser light beam from the light source;
the method comprising:
generating the laser light beam using the light source;
guiding the laser light beam through the optical system so as to exit the device as a treatment laser beam which impinges on an outer surface of the skin to be treated, and
focusing the treatment laser beam, using the optical system, to a single focal spot corresponding to a treatment location in skin tissue;
wherein the method further comprises configuring and arranging the light source to provide a single higher-order laser beam mode, whereby a transverse cross-section of the laser light beam has:
at least a first and a second region of non-zero light intensity, and
a third region, disposed between the first and second regions, of lower light intensity than the non-zero light intensity;
wherein the first and second regions are each configured to create a lesion in skin tissue in the focal spot, and wherein the third region is configured to avoid creating in the focal spot a lesion in skin tissue between the lesions created by the first and second regions.

12. A method according to claim 11, wherein the light source comprises a laser source configured and arranged to oscillate in a transverse mode having a higher order than the fundamental mode of the laser source, wherein the first, second and third regions form part of the intensity pattern of the transverse mode within the transverse cross-section of the laser light beam.

13. Use of a device according to claim 1 in the treatment of skin, in particular non-invasive wrinkle reduction in the skin, reduction of actinic keratoses, scar tissue or acne and reduction of pigmentation spots.
